(19) United States
(12) Patent Application Publication
(10) Pub. No.: US 2026/0263047 A1
(43) Pub. Date: Sep. 10, 2026

(54) Quantum-Processed Ultrasound

(71) Applicant: FUJIFILM SonoSite, Inc., Bothell, WA (US)

(72) Inventors: Thomas Endres, Sagle, ID (US); Craig Chamberlain, Seattle, WA (US); David Knapp, Bellevue, WA (US); Christopher Howard, Seattle, WA (US); Skipper Kagamaster, Boise, ID (US)

(73) Assignee: FUJIFILM SonoSite, Inc., Bothell, WA (US)

(21) Appl. No.: 19/007,231

(22) Filed: Dec. 31, 2024

(51) Int. Cl. A61B 8/00

(52) U.S. Cl. CPC A61B 8/5207; A61B 8/461

(57) ABSTRACT

Techniques and devices are described that implement quantum-processed ultrasound. In example aspects, an ultrasound system includes an ultrasound scanner configured to transmit ultrasound at a patient anatomy and receive reflections of the ultrasound from the patient anatomy. The ultrasound system generates classical ultrasound data based on the received reflections. The ultrasound system is connected to a quantum computing system configured to receive, process, and transmit quantum-formatted data. The classical ultrasound data is converted into quantum ultrasound data for input in the quantum computing system. By performing quantum operations, the quantum computing system produces quantum-processed ultrasound data. The ultrasound system can decode the quantum-processed ultrasound data for use in a classical computing system, such as displaying an ultrasound image.

### BACKGROUND

Ultrasound systems can generate ultrasound images by transmitting sound waves at frequencies above the audible spectrum into a body, receiving echo signals caused by the sound waves reflecting from internal body parts, and converting the echo signals into electrical signals for image generation. Because they are non-invasive and non-ionizing, ultrasound systems are used ubiquitously, such as in emergency departments and point of care. Ultrasound systems rely on classical computer systems and, as such, include or are coupled to a classical computer that processes information represented by bits that assume values of zero and one. Classical computers include an ultrasound machine, a tablet, a smartphone, a server system, and the like. Classical computers perform many tasks much less efficiently than quantum computers, to the extent that some tasks, which are possible for a quantum computer, would be infeasible in a realistic timeframe for a classical computer.

### SUMMARY

Techniques and devices are described that implement quantum-processed ultrasound. In example aspects, an ultrasound system includes an ultrasound scanner configured to transmit ultrasound at a patient anatomy and receive reflections of the ultrasound from the patient anatomy. The ultrasound system generates classical ultrasound data based on the received reflections. The ultrasound system is connected to a quantum computing system configured to receive, process, and transmit quantum-formatted data. The classical ultrasound data is converted into quantum ultrasound data for input in the quantum computing system. By performing quantum operations, the quantum computing system produces quantum-processed ultrasound data. The ultrasound system can decode the quantum-processed ultrasound data for use in a classical computing system, such as displaying an ultrasound image.

Aspects described below include an ultrasound system including an ultrasound scanner configured to transmit ultrasound at a patient anatomy and receive reflections of the ultrasound from the patient anatomy. The ultrasound system further includes a memory configured to store instructions and ultrasound data generated based on the reflections of the ultrasound. The ultrasound system further includes a processor system coupled to the memory and configured to execute the instructions to receive, from the ultrasound scanner, the ultrasound data. The processing system is further configured to execute the instructions to generate quantum-formatted ultrasound data comprising the ultrasound data encoded into a quantum format. The quantum format is configured to be read by a quantum computing system and based on a structure of one or more qubits of the quantum computing system. The processing system is further configured to execute the instructions to transmit the quantum-formatted data to the quantum computing system.

According to some examples, the processing system is further configured to execute the instructions to receive, from the quantum computing system, encoded quantum-processed ultrasound data based on the quantum-formatted ultrasound data and decode the encoded quantum-processed ultrasound data. According to some examples, the ultrasound system further includes a display device. The processing system is further configured to execute the instructions to embed a training object into the ultrasound data and generate, based on the decoded quantum-processed ultrasound data, a first inference for the training object. The processing system is further configured to execute the instructions to generate, based on one or more of the ultrasound data, the decoded quantum-processed ultrasound data, or the first inference, an ultrasound image and cause the display device to display the ultrasound image and a visual representation of the inference.

According to some examples, the ultrasound system further includes an image generator configured to generate an ultrasound image from the reflections of the ultrasound and a data formatter. The data formatter is configured to format the ultrasound image such that it is able to be read by a quantum computing system and based on a structure of one or more qubits of the quantum computing system. The quantum computing system is configured to implement a quantum machine-learned model that is configured to generate, based on the formatted ultrasound image, an inference for the patient anatomy.

Other devices and methods to provide quantum-processed ultrasound are also described. These other devices and methods, in addition to those already disclosed, can be combined to generate additional devices and methods, which, though not explicitly disclosed herein, still are the same in concept as the devices and methods described explicitly herein. The devices and methods explicitly outlined herein are meant to be illustrative and not limiting.

### BRIEF DESCRIPTION OF THE DRAWINGS

The appended drawings illustrate examples and are, therefore, exemplary embodiments and not considered to be limiting in scope.

FIG. 1 illustrates an example environment for a quantum-processed ultrasound device in accordance with one or more implementations.

FIG. 2 illustrates an example ultrasound system for quantum-processed ultrasound in accordance with the present invention.

FIG. 3 illustrates a Bloch sphere showing the concept of encoding qubits in a non-binary state.

FIG. 4 illustrates an example quantum register for quantum-processed ultrasound.

FIG. 5 illustrates an example state preparation for a quantum computing system.

FIG. 6 illustrates a block diagram of an example classical computing device that can perform one or more of the operations described herein, in accordance with some implementations.

FIG. 7 illustrates an example quantum computing device in accordance with the present invention.

FIG. 8 illustrates an example system for embedding a training object into an ultrasound image in accordance with the present invention.

FIG. 9 depicts a method for quantum-processed ultrasound.

FIG. 10 depicts a method for quantum-processed ultrasound.

FIG. 11 depicts a method for quantum-processed ultrasound.

FIG. 12 depicts an example user interface for quantum-processed ultrasound.

FIG. 13 depicts an example ultrasound system for quantum-processed ultrasound.

### DETAILED DESCRIPTION

Classical ultrasound systems can generate ultrasound images by transmitting sound waves at frequencies above the audible spectrum into a body, receiving echo signals caused by the sound waves reflecting from internal body parts, and converting the echo signals into electrical signals for image generation with a classical computing system. Classical computing systems process information represented by bits that can only assume values of zero or one, and such systems are relegated to series processing. Classical computing systems perform many tasks much less efficiently than quantum computing systems, to the extent that some tasks, which are possible for a quantum computing system, would be infeasible in a realistic timeframe for a classical computing system. Even so-called parallel processing classical computing systems are unable to process causally linked processes in parallel.

Unlike classical computing systems, quantum computing systems exploit the quantum principle of superposition to represent information with qubits that can assume not only values of zero and one but also all combinations of these binary values. As such, quantum computing systems can perform computations at rates that will never be achieved by classical computing systems. Using the quantum-mechanical principle of entanglement, a multi-qubit quantum computing system can process multiple causally-linked operations simultaneously, a task that is impossible for a classical computing system. Entangled qubits can also be used to represent all possible bit values in a single state. Quantum computing advantages over classical computing for imaging applications, such as ultrasound, include optimization, numerical analysis, modeling chemical and physical interactions, and applications that do not require fault tolerance, to name a few.

Aspects described below include devices and methods for quantum-processed ultrasound. To allow for quantum-processing of ultrasound, classical and quantum components must both be accessed. For instance, an ultrasound system includes an ultrasound scanner, which can create ultrasound data. Ultrasound data is classical, intended for processing with a classical computing system using binary bits. Bit-coded data is not usable for a quantum computing system. Quantum computing systems exercise quantum-processing on qubits, which are not relegated to strictly binary representations.

### Example Environment

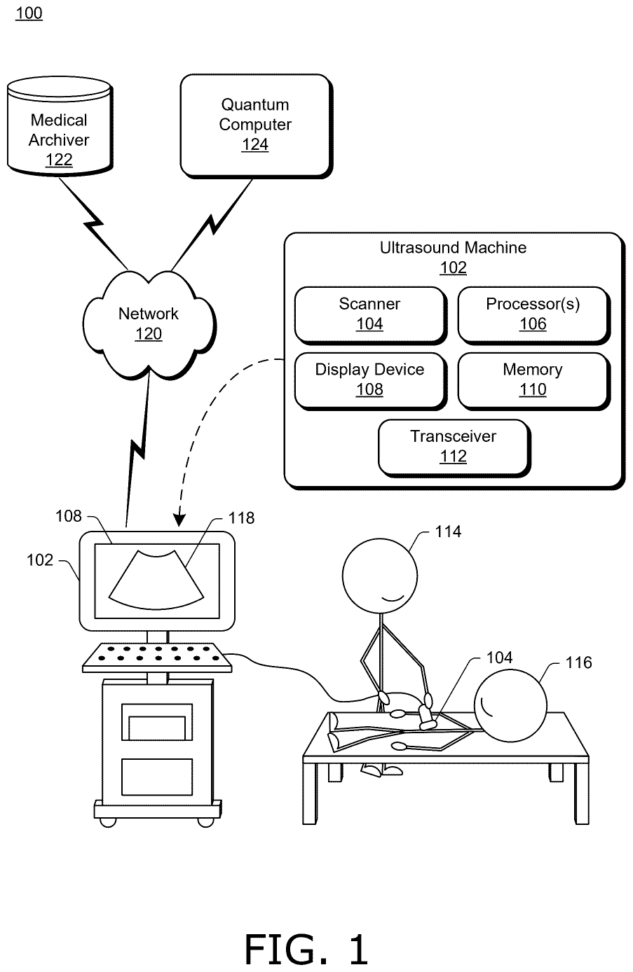

FIG. 1 illustrates an example environment 100 for a quantum-processed ultrasound device in accordance with one or more implementations. The environment 100 includes an ultrasound machine 102 and a scanner 104. The ultrasound machine 102 generates high-frequency sound waves (e.g., ultrasound) and imaging data based on the ultrasound reflecting off a patient's anatomy/body structure, interventional instrument (e.g., needle or catheter), etc. The ultrasound machine 102 includes various components, some of which include the scanner 104, one or more processors 106, a display device 108, a memory 110, and a transceiver 112.

A user 114 (e.g., doctor, nurse, ultrasound technician, operator, sonographer, clinician, etc.) directs the scanner 104 at a patient 116 to non-invasively scan internal bodily structures (e.g., organs, bones, tissue, etc.) of the patient 116 for testing, diagnostic, therapeutic, and/or procedural reasons. In some implementations, the scanner 104 includes an ultrasound transducer array and electronics communicatively coupled to the ultrasound transducer array to transmit ultrasound signals to the patient's anatomy and receive ultrasound signals reflected from the patient's anatomy. In some implementations, the scanner 104 is an ultrasound scanner, which can also be referred to as an ultrasound probe or transducer. In embodiments, the scanner 104 is a multi-array scanner, as described in U.S. patent application Ser. No. 18/613,694 entitled Multi-Dimensional and Multi-Frequency Ultrasound Transducers to Zhang et al. filed Mar. 22, 2024, the disclosure of which is incorporated herein by reference in its entirety. In embodiments, the scanner 104 is a multi-array scanner that includes arrays comprised of lead zirconate titanate (PZT) array elements, capacitive micromachined ultrasonic transducer (CMUT) array elements, and/or piezoelectric micromachined ultrasonic transducer (PMUT) array elements, as described in U.S. patent application Ser. No. 18/957,403 entitled Determining Port Health with Ultrasound to Chamberlain et al. filed Nov. 22, 2024, the disclosure of which is incorporated herein by reference in its entirety.

The display device 108 is coupled to the processor 106, which can include any suitable processor, number of processors, or processor system, such as one or more central processing units, graphics processing units, vector processors, reduced instruction set computing (RISC) processors, complex instruction set computing (CISC) processors, very long instruction word (VLIW) processors, etc. The processor 106 can execute instructions stored on a memory 110 to perform operations disclosed herein for quantum-processed ultrasound. For example, the processor 106 can process the reflected ultrasound signals to generate ultrasound data, including an ultrasound image. Further, the processor 106 can implement one or more machine-learned models (e.g., neural networks) to process the ultrasound data and generate an inference including an object identification, a classification, a label, a segmentation, an additional image (e.g., an image generated in the style of another image), a probability, a score or grade, a recommendation, and the like. Further, the processor 106 can process (e.g., decode) quantum-processed ultrasound data.

The display device 108 is configured to generate and display an ultrasound image (e.g., an ultrasound image 118) of the anatomy and/or an interventional instrument based on the ultrasound data generated by the processor 106 from the reflected ultrasound signals detected by the scanner 104. In some aspects, the ultrasound data includes the ultrasound image 118 or data representing the ultrasound image 118. The transceiver 112 can be configured to transmit (e.g., over a network 120) the ultrasound data and/or any data related to the ultrasound examination, such as medical worksheet data, to a medical archiver 122 (e.g., a vendor neutral archive (VNA)). In embodiments, the transceiver 112 can receive data from the medical archiver 122, such as patient history data or previous examination data.

The transceiver 112 can also be configured to transmit, over the network 120, the ultrasound data and/or a quantum representation of the ultrasound data to a quantum computer 124. In embodiments, the processor 106 implements a data formatter configured to format ultrasound data (e.g., the ultrasound image 118) into quantum-formatted ultrasound data including the ultrasound data encoded into a quantum format. The quantum format is able to be read by the quantum computer 124 and is based on a structure of one or more qubits of the quantum computer 124. The transceiver 112 can transmit the quantum-formatted ultrasound data over the network 120 to the quantum computer 124. The quantum computer 124 can process the quantum-formatted ultrasound data. For example, the quantum computer 124 can implement a quantum machine-learned model that is configured to generate, based on the quantum-formatted ultrasound data, an inference for the patient anatomy. The quantum computer 124 can transmit the inference over the network 120 to the ultrasound machine 102. One or both of the processor 106 and the quantum computer 124 can be configured to transform an inference generated by the quantum computer from a quantum representation to a classical representation, e.g., for display by the display device 108 or further processing by a classically-implemented machine-learned model.

In aspects of quantum-processed ultrasound, the quantum computer 124 is located at a care facility that owns the ultrasound machine 102. In other aspects, the quantum computer 124 is located remotely from the care facility that owns the ultrasound machine 102. In other aspects, the ultrasound machine 102 includes the quantum computer 124. The quantum computer 124 includes two or more qubits, which can be arranged in an initial state based on the quantum-formatted ultrasound data. The quantum computer 124 can process the quantum-formatted ultrasound data by performing quantum operations on the two or more qubits (e.g., the initial state), such as rotations, Pauli operations, or other, linear operations. In some examples, the two or more qubits include one or more other types of quantum information storage, such as qudits, qutrits, etc. In some examples, the two or more qubits are virtual qubits instantiated on a classical computing system. For the purpose of this disclosure, the term qubit is used to represent all quantum computing units of information storage without distinction.

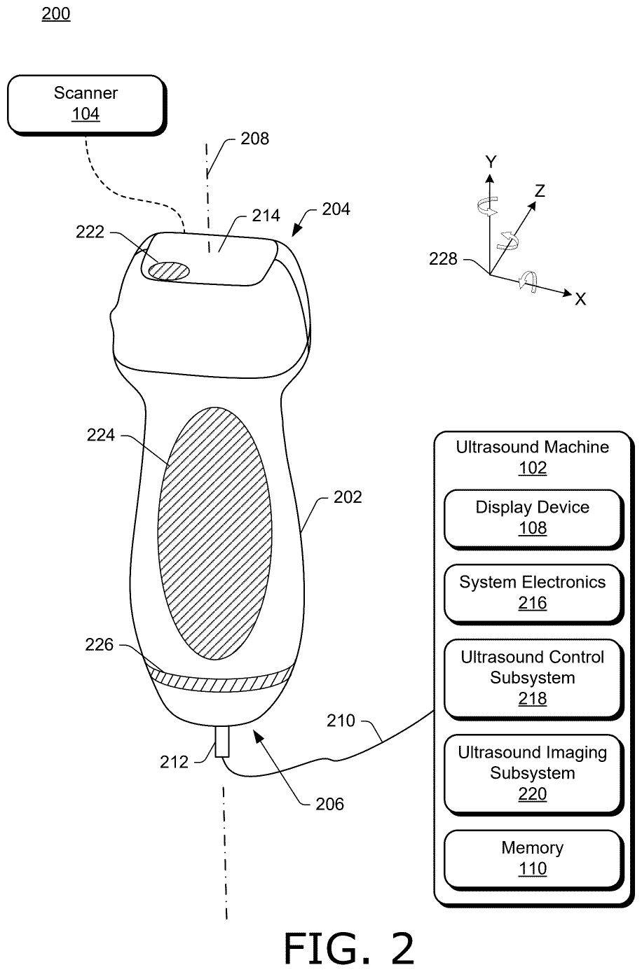

FIG. 2 illustrates an example ultrasound system 200 for quantum-processed ultrasound in accordance with the present invention. The ultrasound system 200 includes the scanner 104 (e.g., ultrasound scanner) that includes an enclosure 202 extending between a distal end portion 204 and a proximal end portion 206. The enclosure 202 includes a central axis 208 (e.g., longitudinal axis) that intersects the distal end portion 204 and the proximal end portion 206. The central axis 208 corresponds to an axial direction of the scanner 104. The scanner 104 is electrically coupled to an ultrasound imaging system (e.g., the ultrasound machine 102) via a coupling 210. In one example, the coupling 210 includes a cable that is attached to the proximal end portion 206 of the scanner 104 by a strain-relief element 212. In some implementations, the coupling 210 includes a wireless coupling so that the scanner 104 is wirelessly coupled to the ultrasound imaging system and communicates with the ultrasound imaging system via one or more wireless transmitters, receivers, or transceivers over a wireless connection or network (e.g., Bluetooth™, Wi-Fi™, etc.).

A transducer assembly 214 having one or more transducer elements is electrically coupled to system electronics 216 in the ultrasound machine 102. In operation, the transducer assembly 214 transmits ultrasound energy from the one or more transducer elements toward a subject and receives ultrasound echoes from the subject. The ultrasound echoes are converted into electrical signals by the transducer element(s) and electrically transmitted to the system electronics 216 in the ultrasound machine 102 for processing and generation of one or more ultrasound images.

Capturing ultrasound data from a subject using a transducer assembly (e.g., the transducer assembly 214) generally includes generating ultrasound signals, transmitting ultrasound signals into the subject, and receiving ultrasound signals reflected by the subject. A wide range of frequencies of ultrasound can be used to capture ultrasound data, such as, for example, low-frequency ultrasound (e.g., less than a low threshold Megahertz (MHz) value) and/or high-frequency ultrasound (e.g., greater than a high threshold MHz value). A particular frequency range to use can readily be determined based on various factors, including, for example, depth of imaging, desired resolution, and so forth.

In some implementations, the system electronics 216 include one or more processors (e.g., the processor(s) 106 from FIG. 1), integrated circuits, application-specific integrated circuits (ASICs), field programmable gate arrays (FPGAs), and power sources to support functioning of the ultrasound machine 102. In some implementations, the ultrasound machine 102 also includes an ultrasound control subsystem 218 having one or more processors. At least one processor, FPGA, or ASIC can cause electrical signals to be transmitted to the transducer(s) of the scanner 104 to emit sound waves and also receive electrical pulses from the scanner 104 that were created from returning echoes. One or more processors, FPGAs, or ASICs can process the raw data associated with the received electrical pulses and form an image that is sent to an ultrasound imaging subsystem 220, which causes the image (e.g., the image 118 in FIG. 1) to be displayed via the display device 108. Thus, the display device 108 displays ultrasound images from the ultrasound data processed by the processor(s) of the ultrasound control subsystem 218.

In some implementations, the ultrasound machine 102 also includes one or more user input devices (e.g., a keyboard, a cursor control device, a microphone, a camera, touchscreen, etc.) that input data and enable taking measurements from the display device 108 of the ultrasound machine 102. The ultrasound machine 102 can also include a disk storage device (e.g., computer-readable storage media such as read-only memory (ROM), a Flash memory, a dynamic random-access memory (DRAM), a NOR memory, a static random-access memory (SRAM), a NAND memory, and so on) for storing the acquired ultrasound data. In aspects, the disk storage device includes the memory 110, which is local to the ultrasound machine 102. Alternatively, the memory 110 used for storing the acquisition data can be remote, such as on a remote server communicatively connected to the ultrasound machine 102. In addition, the ultrasound machine 102 can include a printer that prints the image from the displayed data. To avoid obscuring the techniques described herein, such user input devices, disk storage device, and printer are not shown in FIG. 2.

The scanner 104 in FIG. 2 also includes one or more pressure sensors 222 on a lens of the scanner 104 and one or more pressure sensors 224 on the enclosure 202 of the scanner 104. The pressure sensors 222 and 224 can include in, on, or under a sensor region any suitable type of sensors for determining a pressure. In one example, the pressure sensors 222 and 224 include capacitive sensors that can measure a capacitance, or a change in capacitance, caused by a user's touch or a proximity of touch, as is common in touchscreen technologies. The pressure sensors 222 and 224 can generate sensor data indicative of a touch or a pressure. The sensor data can include a binary indicator that indicates the presence and absence of a touch on the sensor. For instance, a “1” for sensor data can indicate that a pressure is sensed at the pressure sensor, and a “0” for the sensor data can indicate that the pressure is not sensed at the pressure sensor. Additionally or alternatively, the sensor data can include a multi-level indicator that indicates an amount of pressure on the sensor, such as an integer scale from zero to five. For instance, a “0” can indicate that no pressure is detected at the sensor, and a “1” can indicate a small amount of pressure is detected at the sensor. A “2” can indicate a larger amount of pressure is detected at the sensor than a “1”, and a “5” can indicate a maximum amount of pressure is detected at the sensor.

The pressure sensors 222 and 224 are illustrated in FIG. 2 as ellipses for clarity and generally can be of any suitable shape and size. The pressure sensors 222 and 224 generate sensor data indicating pressure at any suitable number of points. In one example, the pressure sensors 222 cover an exterior surface of the lens of the scanner 104 and can be used to determine when the scanner 104 is placed against a patient. Additionally or alternatively, the pressure sensors 224 can substantially cover the enclosure 202 of the scanner 104 and can be used to determine when a clinician grabs the scanner 104 for use in an ultrasound examination (e.g., the clinician has a suitable grip on the scanner 104 to perform the ultrasound examination).

The ultrasound machine 102 can use the sensor data from one or both of the pressure sensors 222 and 224 to generate a trigger signal that can be used for quantum-processed ultrasound. For instance, in some embodiments, when the sensor data from one or both of the pressure sensors 222 and 224 is above a threshold level, and/or the sensor data from the pressure sensors 224 indicates a grip pattern indicative of a human operating the scanner 104, the ultrasound system 200 can generate a trigger signal. The trigger signal can be used to cause a request for ultrasound data to be sent to a quantum computer (e.g., the quantum computer 124 in FIG. 1). For example, the trigger signal can cause the ultrasound data to be encoded into a quantum format and transmitted to the quantum computer (in aspects, generating quantum-formatted ultrasound data, including the ultrasound data encoded into the quantum format). The ultrasound machine 102 can receive quantum-processed ultrasound data from the quantum computer, the quantum-processed ultrasound data based on the encoded ultrasound data and processed by the quantum computer using one or more quantum operators. In some examples, the quantum-processed ultrasound data includes an inference. The display device 108 can display the quantum-processed data, the inference, or an indication of the inference. In embodiments, based on the trigger signal, the system can configure the processor system for encoding of the ultrasound data into a multi-qubit quantum state. For instance, when the trigger signal indicates use of the ultrasound scanner 104, the system can initiate a software routine implemented by the processor system to allocate memory and resources for transformation of the ultrasound data from a classical to a quantum state.

According to some examples, the example ultrasound system 200 requires a quantum format type from the quantum computer in order to encode the ultrasound data. In such examples, the ultrasound machine requests the quantum format from the quantum computer. The ultrasound machine 102 can then receive, from the quantum computer, the quantum format data. Based on the quantum format data, the ultrasound machine 102 can encode the ultrasound data such that it is able to be processed by the quantum computer. In embodiments, the ultrasound machine requests the quantum format from the quantum computer based on the trigger signal, e.g., responsive to the system generating the trigger signal.

In embodiments, the scanner 104 includes an inertial measurement unit (IMU) 226 for generating positional data that determines a position and orientation of the scanner 104 in a coordinate system (e.g., the coordinate system 228 in FIG. 2). The IMU can include a combination of accelerometers, gyroscopes, and/or magnetometers and generate positional data including data representing six degrees of freedom (6DOF), such as yaw, pitch, and roll angles in a coordinate system. Typically, 6DOF refers to the freedom of movement of a body in three-dimensional space. For example, the body is free to change position as forward/backward (surge), up/down (heave), and left/right (sway) translation in three perpendicular axes, combined with changes in orientation through rotation about three perpendicular axes, often termed yaw (normal axis), pitch (transverse axis), and roll (longitudinal axis). For instance, the positional data can indicate that the scanner 104 is within a threshold distance of the patient.

Additionally or alternatively, the example ultrasound system 200 can include a camera and fiducial markers on the scanner 104 (not shown in FIG. 2) to determine the positional data for the ultrasound scanner 104. In one example, the example ultrasound system 200 generates, based on the positional data, a trigger signal, such as the trigger signal previously described, for example, to cause the request for formatting or encoding of the ultrasound data into a quantum format to be read by the quantum computer, and/or for resource allocation for encoding the ultrasound data into the quantum format. For example, the ultrasound data can be encoded into a multi-qubit quantum state to be sent to the quantum computer.

In aspects of ultrasound and quantum computing where the quantum format is not known prior to encoding, the example ultrasound system 200 sends requests to multiple quantum computers for quantum format instructions to determine how to encode the ultrasound data into the quantum format. The example ultrasound system 200 selects one of the multiple quantum computers based on the format instructions received from the quantum computers. For instance, the example ultrasound system 200 can select one of the multiple quantum computers because the ultrasound system 200 is configured to format the ultrasound data to be consistent with the formatting instructions received from that one quantum computer. In another example, the example ultrasound system 200 can select one of the multiple quantum computers based on an availability of the one of the multiple quantum computers.

According to some examples, the multiple quantum computers include multiple types of quantum computation. For example, a first quantum computer of the multiple quantum computers can store quantum image information sufficient to represent the ultrasound data as an ultrasound image and a second quantum computer of the multiple quantum computers can perform one or more quantum-processing algorithms on the encoded ultrasound data, such as spatial rotation, quantum inference, etc. Other examples of quantum computation relevant for quantum-processed ultrasound include, but are not limited to, image compression, edge detection, denoising, encryption, watermarking, image classification, and feature extraction. In aspects of quantum-processed ultrasound data, the example ultrasound system 200 can select the one of the multiple quantum computers based on the type or types of quantum computation the one of the quantum computers is capable of. For example, the ultrasound examination can require a quantum inference to determine a presence of an anomaly within an anatomy of the patient under examination. In such an example, the example ultrasound system 200 can select one of the quantum computers capable of producing a quantum inference for the ultrasound data and subsequently encode the ultrasound data based on received quantum format instructions for the one of the quantum computers capable of producing a quantum inference for the ultrasound data.

### Example Quantum Computing System

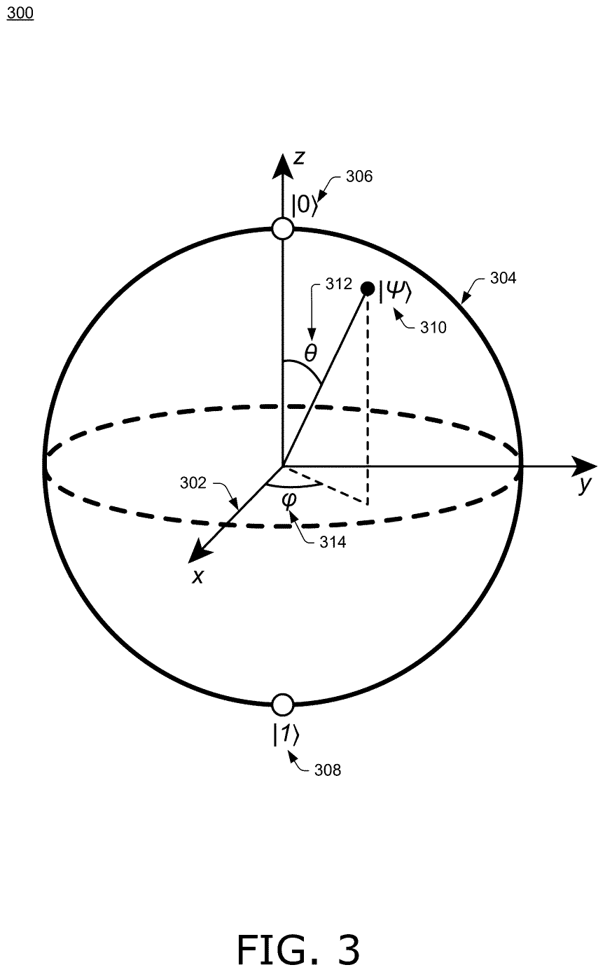

FIG. 3 illustrates a Bloch sphere 300 showing the concept of encoding qubits in a non-binary state in aspects of quantum-processed ultrasound. Classical computing systems encode information in a series of bits, which can take the values of 0 or 1. Advantageously, quantum computing systems, such as the quantum computer 124, use qubits to take on values in-between and inclusive of 0 and 1. The Bloch sphere 300 can be constructed using axes 302, shown here as x-y-z axes in three dimensions. It should be noted that, while three dimensions are shown for clarity, any number of dimensions may be used. In general, qubits are two-dimensional objects but can be represented more succinctly and with mathematical equivalency using the three-dimensional Bloch sphere 300. A normalized sphere 304 is realized a uniform distance of 1 from the origin of the axes 302. At the top of the normalized sphere 304, a first state 306 is found. The first state 306, in Dirac notation, is represented as the |0 [symbol] state 306. The |0 [symbol] state 306, without coefficient (or, equivalently, with a coefficient of 1), is the same as a binary 0. Similarly, at the other pole of the normalized sphere 304, a second state 308 is found. The second state 308, in Dirac notation, is represented as the |1 [symbol] state 308. The |1 [symbol] state 308, without coefficient (or, equivalently, with a coefficient of 1), is the same as a binary 1.

A general state vector 310, represented as the |ψ [symbol] vector 310, represents a qubit superposition state of the |0 [symbol] state 306 and the |1 [symbol] state 308. The |4 [symbol] vector 310 is a state that is in both the |0 [symbol] state 306 and the |1 [symbol] state 308 at the same time. This can be shown in spherical-polar coordinates using an angle θ 312 from the z-axis and an angle φ 314 from the x-axis as follows:

$$\vert ψ 〉 = cos ( \frac{θ}{2} ) \vert 0 〉 + {e}^{i φ} sin ( \frac{θ}{2} ) \vert 1 〉 \qquad Eq . 1$$

Eq. 1 mandates that the |ψ [symbol] vector 310 is normalized. Further, the |0 [symbol] state 306 and the |1 [symbol] state 308 are orthonormal. This is shown mathematically in Eqs. 2 and 3:

$$〈 ψ \vert ψ 〉 = 〈 0 \vert 0 〉 = 〈 1 \vert 1 〉 = 1 \qquad Eq . 2$$

$$〈 m \vert n 〉 = {δ}_{mn} ; m , n ϵ \{ 0 , 1 \} \qquad Eq . 3$$

The symbol δₘₙ in Eq. 3 is the Kronecker Delta. Limits for the angle θ 312 are 0≤θ≤π and limits for the angle φ 314 are 0≤φ≥2π. The following equations can be used to represent the coefficients for the |0 [symbol] state 306 and the |1 [symbol] state 308:

$$cos ( \frac{θ}{2} ) ≡ α \qquad Eq . 4$$

$${e}^{i φ} sin ( \frac{θ}{2} ) ≡ {e}^{i φ} β \qquad Eq . 5$$

The |4 [symbol] vector 310 is a pure state, meaning it ends on a point of the normalized sphere 304. The |ψ [symbol] vector 310 can thus represent not just the |0 [symbol] state 306 and the |1 [symbol] state 308 themselves (which are θ=0 and θ=π, respectively) but also in-between states. This is a marked advantage over a classical, binary computing system, which can only represent the |0 [symbol] state 306 or the |1 [symbol] state 308. In practice, measurement of the |ψ [symbol] vector 310 (as in measurement of a qubit value) will return only the |0 [symbol] state 306 or the |1 [symbol] state 308. Measurement of the coefficients α and β (as well as the phase φ) can be accomplished by multiple measurements of the |ψ [symbol] vector 310, such as by preparing multiple systems including the |ψ [symbol] vector 310 either as copies of one another or by preparing the same system multiple times.

It should be noted that, while the |ψ [symbol] vector 310 has been shown to be a superposition of two states, this is meant to be illustrative and not limiting. Any number of superposition states may be used, including a single, non-superposition state. For example, the angle θ 312 can equal 0, which gives the |ψ [symbol] vector 310 the form of the |0 [symbol] state 306. Other superpositions, including those of a higher dimensionality than 2, can also be represented. The Bloch sphere 300 is used to aid in visualization and conceptualization; other representations can be used with equivalent or different quantum applicability when using qubits of quantum computing systems (e.g., the quantum computer 124).

For example, the |ψ [symbol] vector 310 can be in a state |00 [symbol] , defined as:

$$\vert 00 〉 ≡ \vert 0 〉 ⊗ \vert 0 〉 \qquad Eq . 6$$

In Eq. 6, ⊗ is the tensor operator. The state |00 [symbol] , in some examples, can be a product of multiple vectors. For example, the |ψ [symbol] vector 310 can be combined with another vector |ζ [symbol] to form a third state vector |ω [symbol] ⊗|ψ [symbol] |ζ [symbol] . From combinations such as these, quantum operators (rotation operators, quantum gates, etc.) can be constructed, among other things.

In some aspects of quantum-processed ultrasound data, multiple-qubit states can be entangled. An entangled state |ξ [symbol] is a multiple-qubit state where the constituent qubits are informationally coupled. Mathematically speaking, the state |ξ [symbol] cannot be represented as a tensor product of other states. For example, consider the state |ξ [symbol] of the form:

$$\vert ξ 〉 = \frac{1}{\sqrt{2}} \vert 00 〉 + \frac{1}{\sqrt{2}} \vert 11 〉 \qquad Eq . 7$$

Eq. 7 cannot be expressed by any tensor product combination of state vectors, such as the |ψ [symbol] vector 310 and the |ζ [symbol] vector (|ω [symbol] ). Entangled states (e.g., the state |ξ [symbol] ) have a unique property that a measurement on one of the qubits used to create the entangled state will immediately give the results of all other qubits entangled. For example, consider a qubit q entangled with an ensemble of other qubits [symbol] . Performing a measurement or operation on the qubit q will result in performing the same measurement or operation on all of the qubits in the ensemble [symbol] . This allows for true parallel processing, where qubits causally connected via entanglement are able to process dependent information simultaneously. This is one way in which image and data processing for quantum-processed ultrasound can markedly outperform classical processing for ultrasound data.

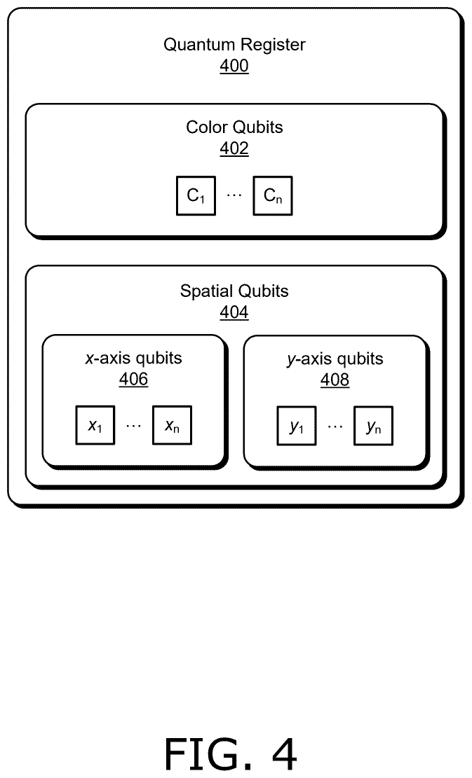

FIG. 4 illustrates an example quantum register 400 for quantum-processed ultrasound. In aspects, the quantum register 400 is a part of a quantum computing system (e.g., the quantum computer 124). The quantum register 400 includes color qubits 402 c₁ through cₙ. The quantum register 400 further includes spatial qubits 404, which for illustration have been separated into x-axis qubits 406 x₁ through xₙ and y-axis qubits 408 y₁ through yₙ.

Consider, for example, an ultrasound image (e.g., the ultrasound image 118) generated by an ultrasound system (e.g., the ultrasound machine 102). In some aspects, the ultrasound image is ultrasound data representing the ultrasound image. In order for the quantum computing system to process the ultrasound image, the ultrasound image must be encoded by the ultrasound system such that it is able to be represented by the qubits of the quantum register 400. In examples where the ultrasound image has spatial and color information, the spatial and color information can be parsed as x-y axis coordinates along with intensity and/or color information for each coordinate, such as by using intensity values, color values, etc. In such an example, the image can be quantum encoded using a variety of techniques, such as a flexible representation of quantum images (FRQI), a flexible representation for quantum color image (FRQCI), multi-channel quantum images (MCQI), a quantum representation model of color digital images (QRCI), etc.

For the purpose of this disclosure, the MCQI technique will be briefly outlined as it applies to ultrasound images. The MCQI technique is used as an illustration and should not be construed as limiting. Any number of other techniques (FRQI, QRCI, etc.) can be used equivalently. The MCQI technique can be used to encode the ultrasound data, where the technique is described in the research paper entitled “An RGB Multi-Channel Representation for Images on Quantum Computers” by Sun et al. In the Journal of Advanced Computational Intelligence and Intelligent Informatics, Vol. 17 No. 3, pp. 404-417, 2013, the disclosure of which is incorporated herein by reference in its entirety.

An image state |I(θ)_{mc} [symbol] (e.g., the |ψ [symbol] vector 310) can be realized in the quantum register 400 from an initial state |0 . . . 0 [symbol] (e.g., a ground state). The image state |I(θ)_{mc} [symbol] can be represented as:

$$\vert {I ( θ )}_{mc} 〉 = \frac{1}{{2}^{n + 1}} {\sum }_{i = 0}^{{2}^{2 n} - 1} \vert {C}_{RGB}^{i} 〉 ⊗ \vert i 〉 \qquad Eq . 8$$

In Eq. 8,

$$\vert {C}_{RGB}^{i} 〉$$

is color information and |i [symbol] is a basis vector. In the quantum register 400, the color qubits 402 are used to represent the color information and the spatial qubits 404 are used to represent the coordinate information. Color and/or intensity values may be encoded in the image state |I(θ)_{mc} [symbol] using amplitude and/or phase values, such as values for intensity. For example, assuming the ultrasound image is an N×N-dimensional RGB image with 24 bits per pixel (8 bits for each color channel), the ultrasound image would classically have 24×N×N bits to represent the ultrasound image in memory. By contrast, the quantum register 400 in this example would have 3 qubits for the color qubits 402 and 2N qubits for the spatial qubits 404 (N of the x-axis qubits 406 and N of the y-axis qubits 408), for a total of 2N+3 qubits used to represent the ultrasound image in memory.

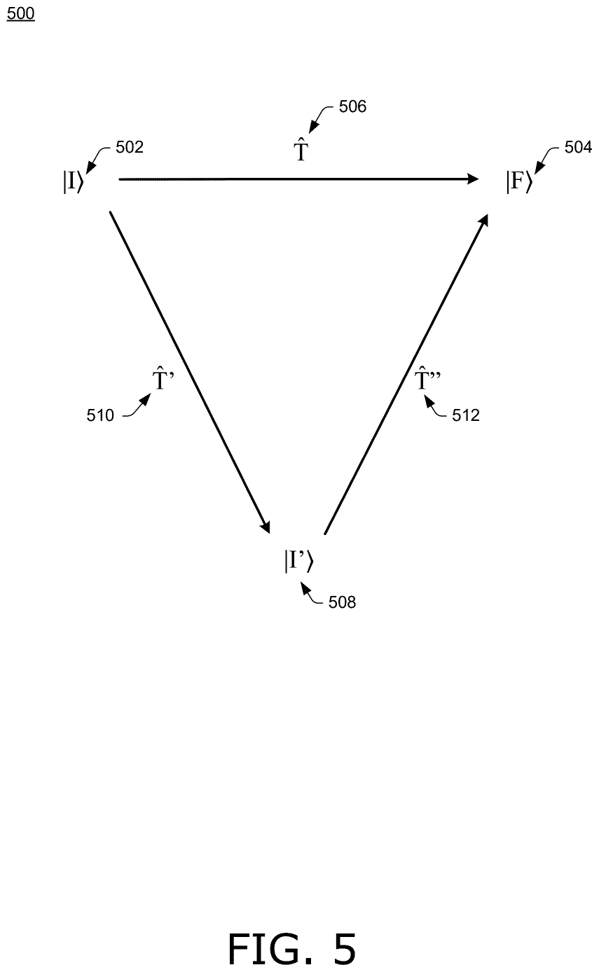

FIG. 5 illustrates an example state preparation 500 for a quantum computing system. In order to quantum-process quantum-encoded ultrasound data, the quantum computing system (e.g., the quantum computer 124) must have a plurality of its qubits transformed from an initial state |I [symbol] 502 (e.g., the |I(θ)_{mc} [symbol] state) to a final state |F [symbol] 504. The final state |F [symbol] 504, which contains the information on the encoded ultrasound image, can be realized by transforming the initial state |I [symbol] 502 using a transformation operator Î 506. This can be shown mathematically as:

$$\hat{T} \vert I 〉 = \vert F 〉 \qquad Eq . 9$$

The transformation operator Î 506 can be any quantum operator or combination of quantum operators. For example, the initial state |I [symbol] 502 of Eq. 9 can be a single qubit, multiple qubits, a mixed quantum state, a quantum entangled state, etc. In examples where the initial state |I [symbol] 502 is the entangled state, the transformation operator Î 506 can be a single transformation operation working on all of the entangled qubits simultaneously. In some examples, the transformation from the initial state |I [symbol] 502 to the final state |F [symbol] 504 proceeds through an intermediary state |I′ [symbol] 508. The transformation from the initial state |I [symbol] 502 to the intermediary state |I′ [symbol] 508 can be shown mathematically as:

$$\vert {I}^{′} 〉 = {\hat{T}}^{′} \vert I 〉 \qquad Eq . 10$$

In Eq. 10, a first intermediary operator Î′ 510 is used. In some examples where the ultrasound image is an n×n-dimensional RGB image, the first intermediary operator Î′ 510 is a Hadamard transformation, such as:

$${\hat{T}}^{′} = I ⊗ {H}^{⊗ 2 n + 2} \qquad Eq . 11$$

In Eq. 11, I is an identity matrix and H is a 2-D Hadamard matrix. In this example, the transformation to the final state |F [symbol] 504 proceeds by performing a second intermediary operator Î″ 512. In examples where the ultrasound image is an n×n-dimensional RGB image and the first intermediary operator Î′ 510 is a Hadamard transformation, the second intermediary operator Î″ is a rotation operator, such as:

$${\hat{T}}^{″} \vert {I}^{′} 〉 = ( {\prod }_{i = 0}^{{2}^{2 n} - I} {R}_{i} ) \vert {I}^{′} 〉 = \vert F 〉 \qquad Eq . 12$$

The Rᵢ in Eq. 12 is comprised of rotation operators in red, green, and blue color spaces, which can be represented mathematically as:

$${R}_{i} = {I}^{⊗ 3} ⊗ {\sum }_{j = 0 , j \neq i}^{{2}^{2 n} - 1} \vert j 〉 〈 j \vert + {R}_{R}^{i} {R}_{G}^{i} {R}_{B}^{i} ⊗ \vert i 〉 〈 i \vert \qquad Eq . 13$$

It should be noted that, while example quantum-encoding and quantum-state preparation methods for quantum-processed ultrasound have been disclosed above, these are not the only methods available to a person skilled in the art. Other methods may be employed without affecting the scope of this disclosure, such as FRQI, FRQCI, QRCI, etc. Additionally or alternately, methods of quantum-processing other than image storage and retrieval can also be equally employed (e.g., quantum methods of image compression, edge detection, denoising, encryption, watermarking, image classification, feature extraction, quantum machine-learned models, etc.).

### Example Devices

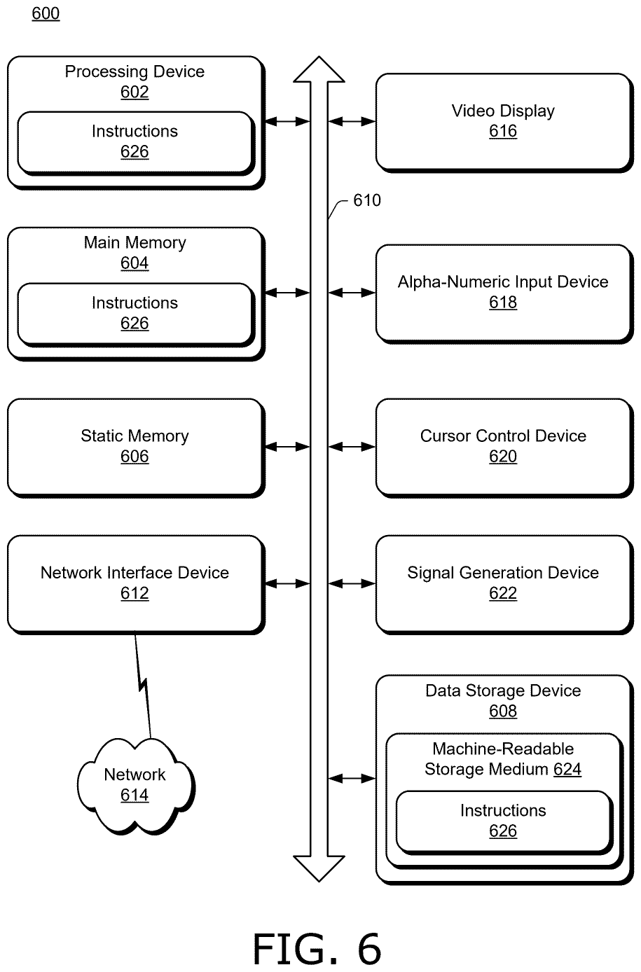

FIG. 6 illustrates a block diagram of an example classical computing device 600 that can perform one or more of the operations described herein, in accordance with some implementations. The classical computing device 600 can be connected to other computing devices in a local area network (LAN), an intranet, an extranet, the Internet, etc. The classical computing device 600 can operate in the capacity of a server machine in a client-server network environment or in the capacity of a client in a peer-to-peer network environment. The classical computing device 600 can be provided by a personal computer (PC), a server computer, a desktop computer, a laptop computer, a tablet computer, a smartphone, an ultrasound machine, combinations thereof, or any machine capable of executing a set of instructions (sequential or otherwise) that specify actions to be taken by that machine. Further, while only a single computing device is illustrated, the term “classical computing device” shall also be taken to include any collection of computing devices that individually or jointly execute a set (or multiple sets) of instructions to perform the methods discussed herein. In some implementations, the classical computing device 600 is one or more of an ultrasound machine, an ultrasound scanner, an access point, a charging station, and a medical archiver.

The example classical computing device 600 can include a processing device 602 (e.g., a general-purpose processor, a programmable logic device (PLD), etc.), a main memory 604 (e.g., synchronous dynamic random-access memory (DRAM), read-only memory (ROM), etc.), and a static memory 606 (e.g., flash memory, a data storage device 608, etc.), which can communicate with each other via a bus 610. The processing device 602 can be provided by one or more general-purpose processing devices such as a microprocessor, a central processing unit, or the like. In an illustrative example, the processing device 602 comprises a CISC microprocessor, a RISC microprocessor, a VLIW microprocessor, or a processor implementing other instruction sets or processors implementing a combination of instruction sets. The processing device 602 can also comprise one or more special-purpose processing devices such as an ASIC an FPGA, a digital signal processor (DSP), a network processor, or the like. The processing device 602 can be configured to execute the operations described herein, in accordance with one or more aspects of the present disclosure, for performing the operations and steps discussed herein.

The classical computing device 600 can further include a network interface device 612, which can communicate with a network 614. The classical computing device 600 also can include a video display unit 616 (e.g., a liquid crystal display (LCD), an organic light-emitting diode (OLED), a cathode ray tube (CRT), etc.), an alphanumeric input device 618 (e.g., a keyboard), a cursor control device 620 (e.g., a mouse), and an acoustic signal generation device 622 (e.g., a speaker, a microphone). In one embodiment, the video display unit 616, the alphanumeric input device 618, and the cursor control device 620 can be combined into a single component or device (e.g., an LCD touch screen).

The data storage device 608 can include a machine-readable storage medium 624 on which can be stored one or more sets of instructions 626 (e.g., instructions for carrying out the operations described herein, in accordance with one or more aspects of the present disclosure). The instructions 626 can also reside, completely or at least partially, within the main memory 604 and/or within the processing device 602 during execution thereof by the classical computing device 600, where the main memory 604 and the processing device 602 also constitute machine-readable media. The instructions 626 can further be transmitted or received over the network 614 via the network interface device 612. For example, the instructions 626 can be instructions for a quantum computer to execute a quantum-processing algorithm on ultrasound data encoded for quantum-processing, with the quantum computer connected to the classical computing device 600 over the network 614.

Various techniques are described in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. In some aspects, the modules described herein are embodied in the data storage device 608 of the classical computing device 600 as executable instructions or code. Although represented as software implementations, the described modules can be implemented as any form of a control application, a software application, a signal-processing and control module, hardware, or firmware installed on the classical computing device 600.

While the machine-readable storage medium 624 is shown in an illustrative example to be a single medium, the term “machine-readable storage medium” should be taken to include a single medium or multiple media (e.g., a centralized or distributed database and/or associated caches and servers) that store the one or more sets of instructions 626. The term “machine-readable storage medium” shall also be taken to include any medium that is capable of storing, encoding, or carrying a set of instructions for execution by the machine and that causes the machine to perform the methods described herein. The term “machine-readable storage medium” shall accordingly be taken to include, but not be limited to, solid-state memories, optical media, and magnetic media.

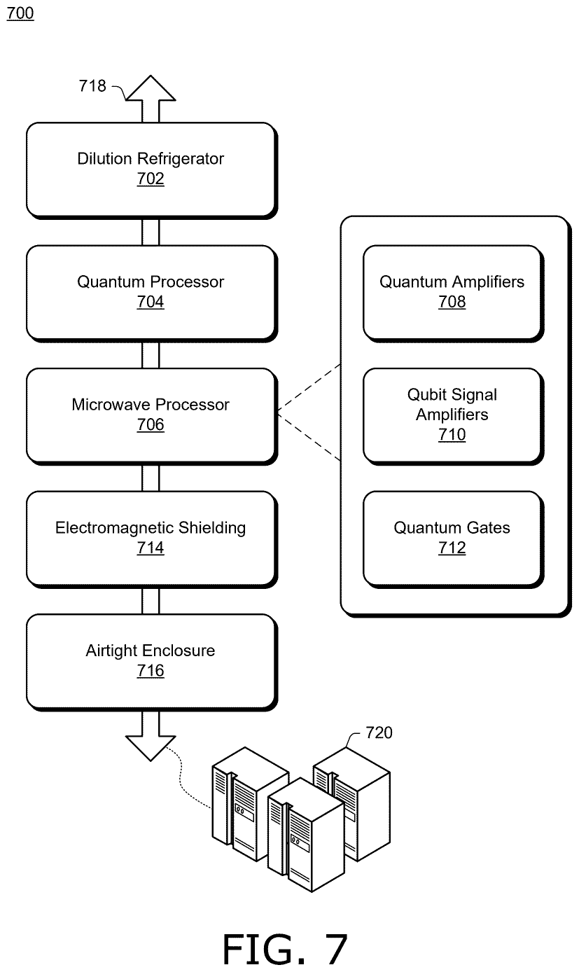

FIG. 7 illustrates an example quantum computing device 700 in accordance with the present invention. As quantum computers can vary in their architectures and implementations, the quantum computing device 700 is meant to be exemplary and non-limiting. For instance, the quantum computing device 700 can include a large-scale system like those currently made by companies including IBM, Google, and Microsoft, an intermediate-scale system as described in “Quantum Computing in the NISQ Era and Beyond” by J. Preskill in Quantum, vol. 2, p. 79, 2018, the disclosure of which is incorporated herein by reference in its entirety, a small-scale system like Google's Willow™ chip architecture, and/or a combination of any of these types and/or scales of systems.

The quantum computing device 700 includes a dilution refrigerator 702, which can circulate a cooling fluid to cool the quantum computing device 700 to temperatures needed to maintain the quantum states of subatomic particles. In an example, the dilution refrigerator 702 includes gold-plated brass to move the cooling fluid, and the cooling fluid is a mixture of helium-3 and helium-4. The dilution refrigerator 702 can cool the quantum computer (e.g., a quantum processor 704 included in the quantum computing device 700) to near absolute zero, such as down to 0.015° Kelvin.

The quantum computing device 700 also includes a microwave processor 706 configured to generate microwave signals that use low-energy pulses to send commands to the quantum processor 704 and read data from the qubits (e.g., the color qubits 402 of FIG. 4, the spatial qubits 404 of FIG. 4). The quantum processor 704 includes one or more of quantum amplifiers 708, qubit signal amplifiers 710, and quantum gates 712. The quantum amplifiers 708 and qubit signal amplifiers 710 amplify quantum signals (e.g., to assist the microwave processor 706 to read the data from the qubits). The quantum gates 712 manipulate the states of the qubits and affect the evolution of the quantum state. Examples of the quantum gates 712 include single-qubit rotation gates, phase shift gates, Pauli gates, and Hadamard gates. The quantum gates 712, in aspects, include any quantum process for quantum-processing of ultrasound data.

In some examples, superconductive circuits of the quantum processor 704 can be extremely sensitive to electromagnetic disturbances. Interaction of the quantum processor 704 with any object outside of the quantum processor 704 can cause pseudo-measurements of the affected qubits (e.g., through wave-function collapse). The quantum computing device 700 also includes electromagnetic shielding 714 that shields the quantum processor 704 from electromagnetic radiation. The quantum computing device 700 also includes an airtight enclosure 716 that can completely surround the quantum computing device 700. The airtight enclosure 716 enables the quantum computing device 700 to be kept in a vacuum and at a temperature close to absolute zero. In an example, the airtight enclosure 716 includes approximately a half-inch thick borosilicate glass that forms an airtight seal.

Components of the quantum computing device 700, including the dilution refrigerator 702, the quantum processor 704, the microwave processor 706, the electromagnetic shielding 714, and the airtight enclosure 716, can be coupled via a bus 718, which can include any suitable coupling, such as microwave cables, electronic circuits, cooling conduits, etc. The quantum computing device 700 is also coupled to a classical computing device 720 (which is an example of the classical computing device 600 in FIG. 6).

The classical computing device 720 can implement control and management and access data management from and to the quantum computing device 700. For instance, the classical computing device 720 can receive encoded ultrasound data in a quantum format from an ultrasound machine and provide an inference generated by the quantum computing device 700 back to the ultrasound machine. In embodiments, the classical computing device 720 can transform an inference generated by the quantum computing device 700 from a quantum representation to a classical representation. Additionally or alternately, the classical computing device 720 can generate a classical inference and compare the classical inference to the inference generated by the quantum computing device 700. In embodiments, the classical computing device 720 includes control electronics for managing the cooling process, controlling the microwave frequencies that manage data input and output, and the like. The quantum computing device 700 can, in some examples, be a standalone device connected to other devices, such as the classical computing device 720, or, in other examples, the quantum computing device 700 can be a part of an overall computing system (including the classical computing device 720, other classical and/or quantum computing devices, etc.).

### Example Quantum Training and Error Determination

Quantum computers often require a strictly maintained environment, with many quantum processors only functional at or around absolute zero (−273° C.), under no atmospheric pressure, and completely insulated from the Earth's magnetic field. Further, retrieving quantum results via observation/measurement can corrupt the data. Moreover, error correction for quantum computers can be difficult, immature, and unreliable. Accordingly, the present invention includes systems, devices, and methods to embed a training object into image data (e.g., ultrasound image data) and use the training object for error correction and detection. The training object has a known property, such as a known label, classification, segmentation, etc. In aspects of quantum-processed ultrasound, an ultrasound system can embed the training object into the image data prior to encoding pixels of the ultrasound image into a quantum format, as described above. The system can then use an output of a quantum-processing by a quantum computer for the training object to determine whether a quantum-processing for a patient anatomy in the image data, such as a quantum inference, is reliable (e.g., accepting or rejecting the quantum inference).

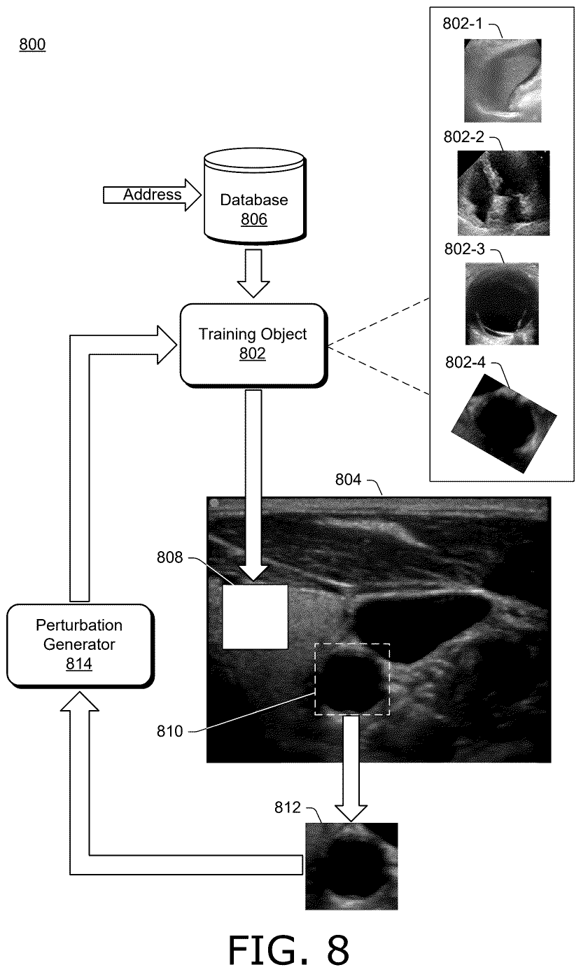

FIG. 8 illustrates an example system 800 for embedding a training object 802 into an ultrasound image 804 in accordance with the present invention. The ultrasound image 804 can be an image, image data, or another representation of ultrasound imagery that can be processed by the system 800. The system 800 can be implemented by an ultrasound system (the ultrasound machine 102, the example ultrasound system 200, etc.), for example, by a processor system of the ultrasound system. The system 800 includes the ultrasound image 804 and the training object 802 that can be embedded into the ultrasound image 804. Examples of the training object 802 illustrated in FIG. 8 include an organ 802-1, a cardiac anatomy 802-2, and an eyeball 802-3. The training object 802 can be maintained in a database 806 of the system 800. The database 806 can be accessed via an address. In an example, the address is randomly generated (e.g., based on a random number or random variable). Additionally or alternatively, the system 800 can select the training object 802 based on an anatomy in the ultrasound image 804, an examination type selected, a protocol, a current step of a protocol, etc.

The system 800 can embed the training object 802 into the ultrasound image 804 at an embed location 808. In an example, the system 800 determines the embed location 808 based on a patient anatomy in the ultrasound image 804. For instance, the system 800 can determine that, at the embed location 808, the ultrasound image 804 does not contain an anatomy of interest. In embodiments, the system 800 determines the embed location 808 with a machine-learned model. In embodiments, the embed location 808 is selected by an operator of the system 800.

Another example of the training object 802 is an anatomy object 802-4. According to some embodiments, the system 800 can generate the anatomy object 802-4 based on a patient anatomy in the ultrasound image 804 by segmenting and extracting the patient anatomy. For example, FIG. 8 includes a bounding box 810 of a blood vessel. The bounding box 810 can represent a segmentation of the blood vessel and be generated with a machine-learned model. The system 800 can extract, based on the bounding box 810, the blood vessel from the ultrasound image 804 to form a segmentation image 812. The segmentation image 812 includes the patient anatomy (i.e., the blood vessel in this example) indicated by the bounding box 810.

The system 800 includes a perturbation generator 814 that can apply a perturbation to the segmentation image 812. Examples of the perturbation include a scaling, a rotation, a stretching, a pinching, a distorting, a removing of a noise or a speckle, a blurring, combinations thereof, and the like. In an example, the anatomy object 802-4 has been generated by the perturbation generator 814 from the segmentation image 812. In aspects of quantum-processed ultrasound, the perturbation generator 814 generates a training object 802 randomly from a segmented anatomy. For example, the system 800 can determine, based on at least one random variable, a perturbation function, segment a patient anatomy in the ultrasound image 804, and apply the perturbation function to the segmented patient anatomy to generate the training object 802. The system 800 can implement a machine-learned model to generate an inference for the training object 802, such as a label, classification, text, etc. In some examples, the machine-learned model is a quantum machine-learned model implemented by a quantum computer.

The training object 802 can be maintained by the database 806 together with inferences (labels, classifications, etc.). Because these inferences are known properties of the training object 802, the system 800 can use them as ground truth values and compare an inference returned by the quantum computer to the known inference for the training object 802. If the two inferences match, then other results returned by the quantum computer for the ultrasound image 804 can be considered reliable by the system 800. In contrast, if the two inferences do not match, then other results returned by the quantum computer for the ultrasound image 804 can be considered unreliable by the system 800. Unreliable results (e.g., inferences) can be discarded by the system 800. In aspects, when an inference for a first ultrasound image is found to be unreliable by the system 800, the system 800 can generate an additional ultrasound image and embed an additional training object into the additional ultrasound image. The system 800 can then generate an encoded additional ultrasound image based on a quantum format for the additional ultrasound image and request an additional inference for the encoded additional ultrasound image from the quantum computer. The system 800 can repeat these steps until the system 800 is able to match inferences for the training object 802 and make a determination that the ultrasound image is reliable.

In some examples, the unreliable results and/or the reliable results can be used as labeled data sets for machine training purposes (training the machine-learned model, training the quantum machine-learned model, etc.). For example, a training data set can include data such as patient data from an ultrasound examination. Unreliable results and/or reliable results from the ultrasound examination can, in some examples, be used as inputs for the machine-learned model, the quantum machine-learned model, etc. For example, a model (the machine-learned model, the quantum machine-learned model, etc.) can be trained with a supervised algorithm to match results/inferences to labels on the inputs (e.g., a label of reliable, a label of unreliable). Further, in some examples, the model can be retrained, tuned, fine-tuned, augmented, etc. by new data from a new ultrasound examination of the patient.

In some examples, the inferences are associated with one or more reliability measures. For example, one or more of the reliability measures can be threshold values. In another example, the one or more reliability measures can be normalized reliability values from 0 to 1. In aspects, the reliability measures represent a confidence in the veracity, utility, or applicability of the inferences. If the reliability measure for an inference is below a threshold value, then the system 800 can reject the inference and request another one from the quantum computer.

### Example Methods

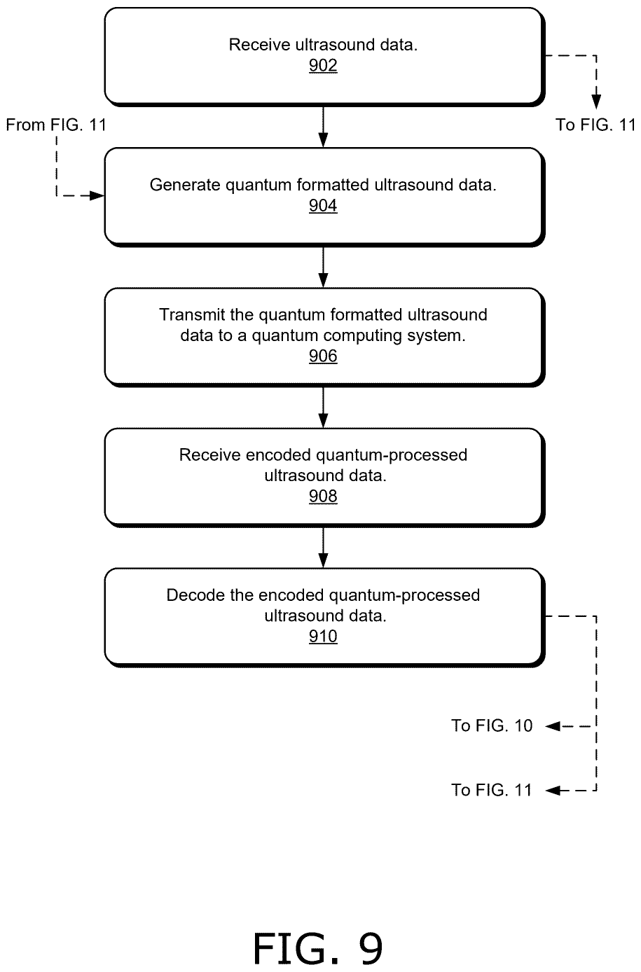

FIGS. 9-11 depict various methods 900-1100, respectively, for quantum-processed ultrasound. The methods 900-1100 are shown as sets of blocks that specify operations performed but are not necessarily limited to the order or combinations shown for performing the operations by the respective blocks. Further, any of one or more of the operations can be repeated, combined, reorganized, or linked to provide a wide array of additional and/or alternate methods. In portions of the following discussion, reference can be made to the example environment 100 of FIG. 1 or to entities or processes as detailed in FIGS. 2-8, reference to which is made for example only. The techniques are not limited to performance by one entity or multiple entities operating on one device. The methods 900-1100 can be performed by an ultrasound machine, such as the ultrasound machine 102 of FIG. 1. Further, one or more operations from any of the methods 900-1100 can be combined with one or more operations from any other of the methods 900-1100 to generate additional and/or alternate methods, which are directly inferred from this disclosure.

FIG. 9 depicts the method 900 for quantum-processed ultrasound. At 902, ultrasound data is received. In aspects, the ultrasound data is received by an ultrasound scanner (e.g., the scanner 104 of FIG. 1). In aspects, the ultrasound scanner is configured to transmit ultrasound at a patient anatomy and receive reflections of the ultrasound from the patient anatomy, and the received ultrasound data is based on the reflections of the ultrasound from the patient anatomy. In some examples, the ultrasound data is image data, the image data including information on one or more of intensity values, location values, or color values configured to construct one or more pixels from the image data.

At 904, quantum-formatted ultrasound data is generated by encoding the ultrasound data into a quantum format. In aspects, the quantum format is configured to be read by a quantum computing system and based on a structure of one or more qubits of the quantum computing system. According to some examples where the ultrasound data is image data, the encoding of the image data into the quantum format includes mapping one or more of the intensity values, the location values, or the color values to one or more of an amplitude value or a phase value for one or more of the one or more qubits of the quantum computing system. In some examples, the one or more qubits of the quantum computing system are virtual qubits instantiated on a classical computing system.

At 906, the quantum-formatted ultrasound data is transmitted to the quantum computing system. In aspects, the method 900 is executed by an ultrasound system, the ultrasound system including the ultrasound scanner, a memory configured to store instructions and ultrasound data generated based on the reflections of the ultrasound, and a processor system coupled to the memory and configured to execute the instructions. In aspects, the instructions are such that they include the steps of the method 900. In some implementations, the ultrasound system is located at a care facility and the quantum computing system is located remotely from the care facility. In some embodiments, the ultrasound system and the quantum computing system are located in a same facility. In some examples, the ultrasound system includes the quantum computing system.

At 908, encoded quantum-processed ultrasound data is received. In aspects, the encoded quantum-processed ultrasound data is based on the quantum-formatted ultrasound data. In some examples, the encoded quantum-processed ultrasound data includes the quantum-formatted ultrasound data processed by one or more quantum operations. The one or more quantum operations are configured to transform one or more initial states of the one or more qubits into one or more transformed states of the one or more qubits. In some examples, the structure of the one or more qubits of the quantum computing system includes an entanglement of at least two of the one or more qubits of the quantum computing system, and at least one of the one or more quantum operations transforms the two or more entangled qubits of the quantum computing system in a single transformation operation.

At 910, the encoded quantum-processed ultrasound data is decoded. In examples where the encoded quantum-processed ultrasound data includes the quantum-formatted ultrasound data processed by one or more quantum operations, the decoded quantum-processed ultrasound data is based on the one or more transformed states of the one or more qubits. In aspects, the decoding of the encoded quantum-processed ultrasound data is performed by a classical computing system of the ultrasound system (e.g., the classical computing device 720).

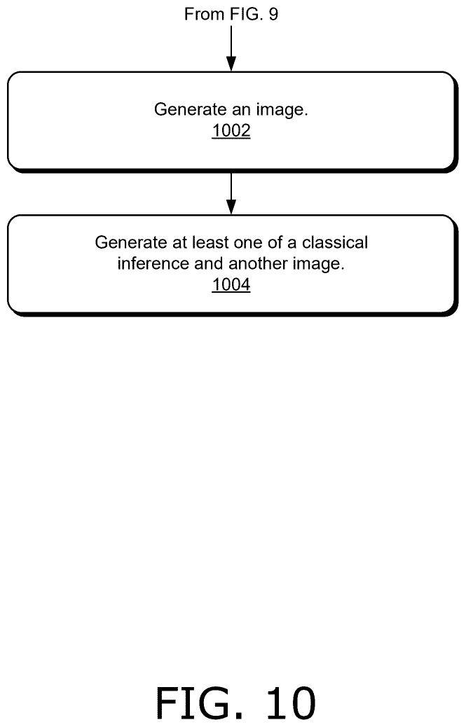

FIG. 10 depicts the method 1000 for quantum-processed ultrasound. At 1002, proceeding from 910 in FIG. 9, an image is generated. In aspects, the image is generated based on the decoded quantum-processed ultrasound data. According to some examples, the decoded quantum-processed ultrasound data includes a quantum inference based on the ultrasound data. In some implementations, the quantum computer is configured to implement a quantum machine-learned model to generate the quantum inference.

At 1004, at least one of a classical inference and another image is generated. In aspects, the at least one of the classical inference and the other image is generated by a machine-learned model and is based on the decoded quantum-processed ultrasound data. According to some examples, a display device is implemented to display a comparison of the quantum inference and the classical inference or a comparison of the image and the other image.

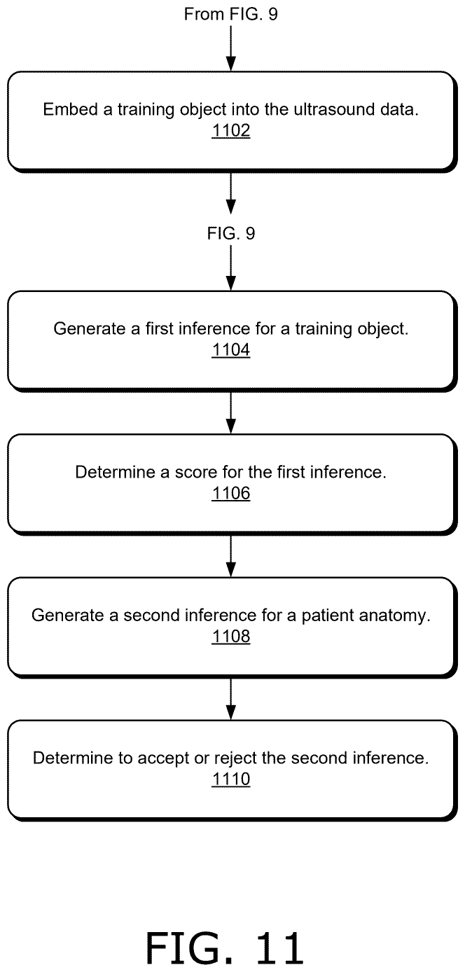

FIG. 11 depicts the method 1100 for quantum-processed ultrasound. At 1102, proceeding from 902 of FIG. 9, a training object is embedded into the ultrasound data. In some examples, the training object is selected by the ultrasound system from among a plurality of available training objects, such as by an operator of the ultrasound system. According to some examples, the training object is generated by the ultrasound system. For instance, a machine-learned model can be used by one or more processors of the ultrasound system to generate the training object.

At 1104, proceeding from 910 of FIG. 9, a first inference for the training object is generated. The generation of the first inference for the training object is based on the decoded quantum-processed ultrasound data and the embedded training object. In some examples, the generation of the first inference is performed by the machine-learned model. At 1106, a score for the first inference is determined. In aspects, the score for the first inference is based on the training object.

At 1108, a second inference for the patient anatomy is generated. In aspects, the second inference for the patient anatomy is based on the decoded quantum-processed ultrasound data. At 1110, a determination is made to accept or reject the second inference for the patient anatomy. In aspects, the determination is based on the score for the first inference and/or a comparison between the first inference and the second inference.

### Example User Interface

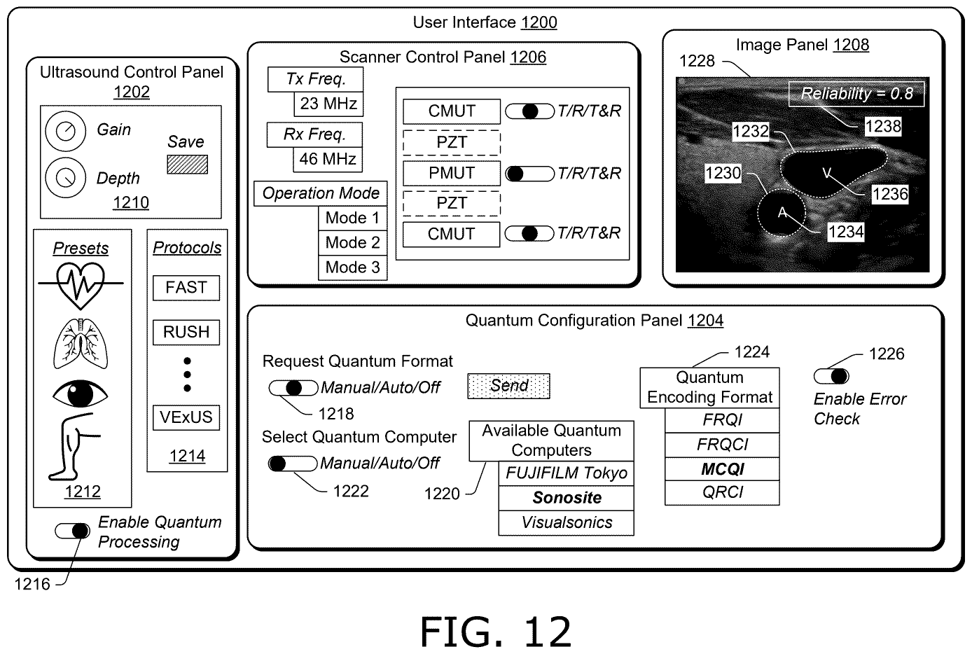

FIG. 12 illustrates some embodiments of a user interface 1200 of a system for quantum-processed ultrasound. The user interface can be displayed on any suitable computing device of the system for quantum-processed ultrasound, including one or more of the ultrasound machine 102, the classical computing device 600, and the classical computing device 720. The user interface 1200 includes an ultrasound control panel 1202, a quantum configuration panel 1204, a scanner control panel 1206, and an image panel 1208.

The ultrasound control panel 1202 can include any suitable controls for configuring an ultrasound system for quantum-processed ultrasound. In the example in FIG. 12, the ultrasound control panel 1202 includes ultrasound controls 1210 for adjusting gain and depth and saving an image. The ultrasound control panel 1202 also includes controls 1212 for selecting examination presets. The controls 1212 for examination presets are represented by selectable icons for a cardiac examination, a respiratory examination, an ocular examination, and a muscular-skeletal examination. These examination presets, when selected, can configure the ultrasound machine with predetermined values of gain and depth, and/or other imaging parameters (e.g., beamformer settings, filter coefficients, amplitude settings, etc.).

The ultrasound control panel 1202 also includes controls 1214 for selecting ultrasound protocols, for example including a Focused Assessment with Sonography for Trauma (FAST) protocol, a Rapid Ultrasound for Shock and Hypotension (RUSH) protocol, and a Venous Congestion Evaluation using Ultrasound (VExUS) protocol. In some examples, other ultrasound protocols not pictured can be included. A user can select one of these example protocols, and in response, the system can configure itself for an examination in accordance with the protocol, including to display a protocol panel in the user interface 1200 (not shown for clarity) with guided steps needed to complete the selected protocol.

The ultrasound control panel 1202 also includes a selection 1216 (e.g., an electronic rocker switch) to enable quantum processing of ultrasound data. Responsive to the selection 1216 to enable quantum processing of ultrasound data (e.g., via the rocker switch in the ultrasound control panel 1202), the user interface 1200 displays the quantum configuration panel 1204. The quantum configuration panel 1204 can display any suitable option, control, or setting to configure quantum-processed ultrasound. In the example in FIG. 12, the quantum configuration panel 1204 includes a three-way switch 1218 to request a quantum format, e.g., from a quantum computer, such as a quantum computer selected via the pull-down menu 1220. The three-way switch 1218 can be set to a manual position, an automatic position, and an off position. In the example in FIG. 12, the three-way switch 1218 is in the automatic position. When the three-way switch 1218 is in the automatic position, the system can automatically send a request to a quantum computer for a quantum format to encode ultrasound data for quantum processing, e.g., automatically responsive to a trigger signal generated by the system, as previously described. For instance, the trigger signal can be generated based on sensor data from one or more pressure sensors, IMU data, etc. A send button next to the three-way switch 1218 is modified, such as filled with a dotted pattern in FIG. 12, to indicate the send button is not available for use, because the three-way switch 1218 is in the automatic position. In embodiments, if the three-way switch 1218 was in the manual position, the user interface 1200 displays the send button so that is not greyed out, and is selectable. Hence, when the three-way switch 1218 is in the manual position, a request for a quantum format can be sent to a quantum computer responsive to the send button being activated, e.g., touched.

The quantum configuration panel 1204 also includes a three-way switch 1222 to select an available quantum computer. When the three-way switch 1222 is in the manual position, the quantum configuration panel 1204 can display the pull-down menu 1220 that includes available quantum computers. In the example in FIG. 12, the pull-down menu 1220 includes quantum computers named FUJIFILM Tokyo, Sonosite, and Visualsonics. The quantum computer Sonosite has been selected (e.g., by the user), as evidenced by its designation in bold in the pull-down menu 1220. When the three-way switch 1222 is in the automatic position, the system can automatically select a quantum computer, e.g., one of the quantum computers included in the pull-down menu 1220. The system can automatically select one of the quantum computers based on any suitable criteria, including a selected examination preset, a selected protocol, an anatomy detected in an ultrasound image (e.g., with a machine-learned model), the quantum format enabled by the system for quantum processing, and the like. When the three-way switch 1222 is in the off position, the system can select a default quantum computer for quantum processing of ultrasound data, such as a quantum computer of the care facility in which the ultrasound machine is located and the ultrasound examination is performed.

The quantum configuration panel 1204 also includes a pull-down menu 1224 that includes quantum encoding formats for encoding ultrasound data for quantum processing. In the example in FIG. 12, the pull-down menu 1224 includes quantum encoding formats FRQI, FRQCI, MCQI, and QRCI. The MCQI format is enabled, as evidenced by its designation in bold in the pull-down menu 1224. The pull-down menu 1224 can be used to select a quantum encoding format when the three-way switch 1218 is in the off position, e.g., when a request for the quantum encoding format is not sent to a quantum computer. Additionally or alternatively, the pull-down menu 1224 can indicate a quantum encoding format for which the system is enabled based on a request sent to a quantum computer when the three-way switch 1218 is in the manual or automatic positions.

The quantum configuration panel 1204 also includes a switch 1226 to enable error checking of quantum data returned by a quantum computer, as described previously with respect to FIG. 8. For example, when error checking is enabled via the switch 1226, the system can insert a training object (e.g., the training object 802) into an ultrasound image, and compare an inference generated by a quantum computer for the training object to a known, ground truth inference for the training object. Based on this comparison, the system can generate a reliability measure for the inference and determine whether to accept or reject the inference or other data generated by the quantum computer based on the ultrasound image with the inserted training object.

The scanner control panel 1206 includes controls and selections for configuring one or more arrays of a transducer assembly of an ultrasound scanner for quantum-processed ultrasound. In some embodiments, the scanner control panel 1206 includes options to select transmit and receive frequencies for one or more arrays of an ultrasound scanner. In the example in FIG. 12, a transmit frequency is set via a drop-down menu to 23 MHz, and a receive frequency is set via a drop-down menu to 46 MHz. In some embodiments, the scanner control panel 1206 also includes options for enabling and configuring one or more arrays of a multi-array transducer. In the example in FIG. 12, the multi-array scanner includes five arrays (or sub-arrays), including a center array comprised of PMUT array elements, two adjacent arrays comprised of PZT array elements, and two outer arrays comprised of CMUT array elements. As indicated by the dashed lines, the PZT arrays are disabled, and as indicated by the solid lines, the PMUT and CMUT arrays are enabled. In some embodiments, a user can enable and disable an array by touching or tapping on the visual representation for the array. The scanner control panel 1206 also includes options (e.g., three-position electronic rocker switches) to configure the enabled arrays for transmission, reception, or both transmission and reception. In the example in FIG. 12, the PMUT array (e.g., the center array) is configured to transmit, and the CMUT arrays (e.g., the outer arrays) are configured to receive. In some embodiments, the PMUT arrays have better transmit sensitivity (in terms of power efficiency) than the CMUT arrays, while the CMUT arrays have better receive sensitivity (in terms of signal strength) than the PMUT arrays.

In some embodiments, the scanner control panel 1206 also includes a drop-down menu to enable the ultrasound scanner according to an operation mode. Example operation modes include Mode 1 which can enable at least one array of the ultrasound scanner as a linear array and at least one additional array of the ultrasound scanner as a phased array, Mode 2 which can enable the arrays for broadband tissue harmonic imaging (broadband THI), and Mode 3 which can enable the arrays for full-aperture broadband THI operation.

The image panel 1208 can display any suitable images, e.g., ultrasound images, inferences, and/or visual representations for quantum-processed ultrasound. In the example in FIG. 12, the image panel 1208 displays an ultrasound image 1228, which is an example of an ultrasound image that can be generated by an ultrasound machine, e.g., the ultrasound machine 102. The ultrasound image 1228 includes two blood vessels. A first blood vessel is outlined by a segmentation 1230, and a second blood vessel is outlined by a segmentation 1232. One or more of the segmentations 1228, 1230 can be generated by a classical computing device, e.g., the classical computing device 600. Additionally or alternatively, one or more of the segmentations 1228, 1230 can be generated by a quantum computing device, e.g., the quantum computing device 700. The blood vessels also include labels that classify them as veins or arteries. For example, the first blood vessel that is outlined by the segmentation 1230 is labeled by label 1234 (an “A”) that designates the first blood vessel as an artery. The second blood vessel that is outlined by the segmentation 1232 is labeled by label 1236 (an “V”) that designates the second blood vessel as n vein. One or more of the labels 1234, 1236 can be generated by a classical computing device, e.g., the classical computing device 600. Additionally or alternatively, one or more of the labels 1234, 1236 can be generated by a quantum computing device, e.g., the quantum computing device 700.

In an example, the ultrasound image 1228 includes a training object (e.g., the training object 802). For instance, one of the blood vessels can be the training object. The system can compare an inference (e.g., one of the labels 1234, 1236) generated by a quantum computer for the training object to a ground truth inference for the training object, and, based on this comparison, generate a reliability measurement. The reliability measurement can indicate a reliability value for another inference made by the quantum computer. For example, the system can compare the label 1234 for the first blood vessel generated by a quantum computer to a ground truth value for the label, and, based on the comparison, generate the reliability measurement for the label 1236 of the second blood vessel. The image panel 1208 can display a visual representation of the reliability measurement, such as the text 1238, that indicates a reliability measurement of 0.8, e.g., 80% confidence value.

### Example Ultrasound System

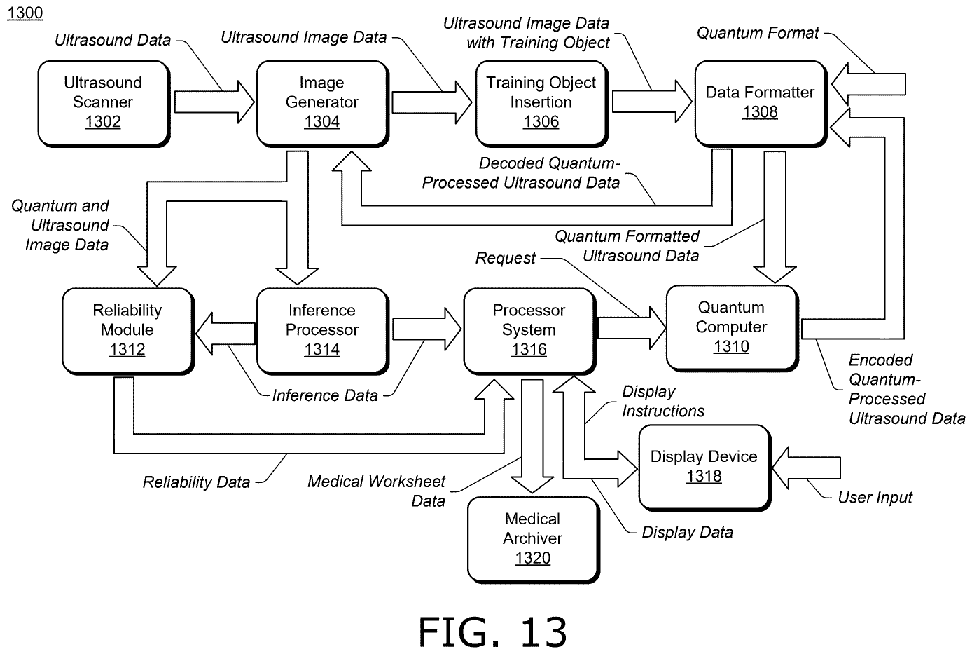

FIG. 13 illustrates an example ultrasound system 1300 for quantum-processed ultrasound in accordance with some embodiments. The ultrasound system 1300 is an example of the ultrasound machine 102 illustrated in the environment 100 of FIG. 1 and the example ultrasound system 200 in FIG. 2. The ultrasound system 1300 includes an ultrasound scanner 1302, which is an example of the scanner 104 as previously described. The ultrasound system 1300 also includes an image generator 1304, a training object insertion block 1306, a data formatter 1308, a quantum computer 1310, a reliability module 1312, an inference processor 1314, a processor system 1316, a display device 1318, and a medical archiver 1320.

The ultrasound scanner 1302 transmits ultrasound at a patient anatomy and generates, based on the ultrasound reflections from the patient anatomy, ultrasound data. The ultrasound scanner 1302 provides the ultrasound data to the image generator 1304. The image generator 1304 can generate any suitable image from the ultrasound data, e.g., ultrasound image data, such as a B-mode image, M-mode image, segmentation image (e.g., an image with a segmentation of the patient anatomy), and the like. The image generator 1304 provides the ultrasound image data to the training object insertion block 1306, the reliability module 1312, and the inference processor 1314.

The training object insertion block 1306 can be implemented to insert one or more training objects into an ultrasound image (e.g., the ultrasound image data) to generate ultrasound image data with a training object. For instance, the ultrasound image 804 in FIG. 8 is an example of ultrasound image data with a training object generated by the training object insertion block 1306. Further, the system 800 in FIG. 8 is an example of the training object insertion block 1306. The training object insertion block 1306 provides the ultrasound image data with a training object to the data formatter 1308.

The data formatter 1308 can implement an encoder to encode the ultrasound image data with a training object from the training object insertion block 1306 according to a quantum format. In aspects, the data formatter 1308 generates quantum-formatted ultrasound data by encoding the ultrasound image data with the training object into the quantum format. In embodiments, the quantum format is configured to be read by the quantum computer 1310 and is based on a structure of one or more qubits of the quantum computer 1310. The data formatter 1308 can obtain the quantum format based on a request sent by the processor system 1316 to the quantum computer 1310. Additionally or alternatively, the data formatter 1308 can obtain the quantum format based on a user selection in a user interface (e.g., the user interface 1200) implemented on the display device 1318. The quantum-formatted ultrasound data generated by the data formatter 1308 is provided to the quantum computer 1310.

The quantum computing device 700 is an example of the quantum computer 1310. The quantum computer 1310 receives the quantum-formatted ultrasound data from the data formatter 1308 and generates encoded quantum-processed ultrasound data. For instance, the quantum computer 1310 can generate the encoded quantum-processed ultrasound data by implementing one or more quantum operations to transform one or more initial states of the one or more qubits into one or more transformed states of the one or more qubits. In embodiments, the quantum computer 1310 implements one or more quantum machine-learned models to generate a quantum inference (e.g., label, segmentation, classification, image, etc.). The quantum inference can be included in the encoded quantum-processed ultrasound data. The quantum computer 1310 provides the encoded quantum-processed ultrasound data to the data formatter 1308.

The data formatter 1308 can implement a decoder configured to decode the encoded quantum-processed ultrasound data generated by the quantum computer 1310 and generate decoded quantum-processed ultrasound data. The decoder can be based on the quantum format obtained by the data formatter 1308. The decoded quantum-processed ultrasound data can be represented in a format readable to a classical computing system. The data formatter 1308 provides the decoded quantum-processed ultrasound data to the image generator 1304.

The image generator 1304 receives the decoded quantum-processed ultrasound data from the data formatter 1308 and can generate quantum image data from it. For instance, the quantum image data can be represented as pixel data (e.g., location data with color and/or intensity data) that is based on the encoded quantum-processed ultrasound data generated by the quantum computer 1310. The image generator 1304 provides the quantum image data, along with the ultrasound image data, to the reliability module 1312 and the inference processor 1314.

The reliability module 1312 receives the quantum image data and the ultrasound image data from the image generator 1304, as well as inference data from the inference processor 1314. The reliability module 1312 can generate any suitable reliability measure, score, grade, and the like to determine a reliability (or confidence level) of data generated by the quantum computer 1310. In an example, the inference data includes two inferences generated based on data generated by the quantum computer 1310. For instance, the inference processor 1314 can generate, based on the decoded quantum-processed ultrasound data and the embedded training object, a first inference for the training object and a second inference for a patient anatomy. The reliability module 1312 can determine, based on the training object, a score for the first inference, and, based on the score for the first inference, determine to accept or reject the second inference for the patient anatomy. The reliability module 1312 can include a reliability measure, score, acceptance/rejection determination, and the like in reliability data that is provides to the processor system 1316.

The inference processor 1314 receives the quantum image data and the ultrasound image data from the image generator 1304 and can generate any suitable inference from this data. Examples of inferences include segmentations, labels, classifications, quality scores, recommendations, etc. In embodiments, the inference processor 1314 implements one or more machine-learned models to generate an inference. In an example, the inference processor 1314 represents an inference generated by the quantum computer 1310 as pixel data (e.g., location data with color and/or intensity data). The inference processor 1314 provides the inference data (e.g., data representing one or more inferences) to the reliability module 1312 and the processor system 1316.

The processor system 1316 receives the reliability data from the reliability module 1312 and the inference data from the inference processor 1314, as well as display instructions from the display device 1318. For instance, the display instructions can be based on a user input received via the user interface implemented on the display device 1318. The user input can indicate user selections that designate what types of data the user wants displayed on the display device 1318. Further, although not shown for clarity, the processor system 1316 can access any data passed between blocks of the ultrasound system 1300 and any intermediate results generated by the blocks of the ultrasound system 1300. Based on the reliability data, the inference data, the user display instructions (and in embodiments, the quantum image data and the ultrasound image data), the processor system 1316 generates display data that includes data to be displayed on the display device 1318. For example, the content displayed in the user interface 1200 can be the display data generated by the processor system 1316. The processor system 1316 provides the display data to the display device 1318, which can be implemented to display the display data, e.g., via the user interface.

In aspects of quantum-processed ultrasound, the display device 1318 is implemented to display a comparison of a quantum inference generated by the quantum computer 1310 and a classical inference generated by the inference processor 1314. Additionally or alternatively, the display device 1318 can display a comparison of two images including a classically-generated image and a quantum-generated image, such as a first image generated based on data from the quantum computer 1310 (e.g., from the quantum image data), and a second image generated based on the ultrasound data (e.g., the ultrasound image data).

The processor system 1316 can also send a request to the quantum computer 1310 for a quantum format to encode the ultrasound data (e.g., the ultrasound data, the ultrasound image data and/or the ultrasound image data with training object). In an example, the processor system 1316 sends the request to the quantum computer 1310 automatically, based on a trigger signal generated by the ultrasound scanner 1302 that indicates a grip pattern indicative of a human operating the ultrasound scanner 1302, or use of the ultrasound scanner 1302 to perform an ultrasound examination.

In embodiments, the processor system 1316 is configured to populate a medical worksheet with data obtained as part of an ultrasound examination with the ultrasound system 1300, such as an indication of an inference for a patient anatomy. The populating can be conditioned upon a determination made by the reliability module 1312 to accept the inference for the patient anatomy. The processor system 1316 cause a transceiver to send medical worksheet data, including the medical worksheet populated with the indication of the inference, to the medical archiver 1320.

### CONCLUSION

Aspects of quantum-processed ultrasound as described herein are advantageous, as they provide for benefits such as decreased storage usage for ultrasound image storage and retrieval, improved image compression, edge detection, denoising, encryption, watermarking, image classification, feature extraction, and quantum machine-learned model implementation, to name a few. The techniques of quantum-processed ultrasound disclosed herein also aid in generating quantum inferences based on ultrasound data, which may not be available in a classical computer-based ultrasound processing. The quantum-processed ultrasound provides increased scanning efficiency, improved patient experience, higher-fidelity scanning outcomes, and similar benefits.

While the present subject matter has been described in detail with respect to various specific example implementations thereof, each example is provided by way of explanation and not limitation of the disclosure. Those skilled in the art, upon attaining an understanding of the foregoing, can readily produce alterations to, variations of, and equivalents to such implementations. Accordingly, the subject disclosure does not preclude inclusion of such modifications, variations, and/or additions to the present subject matter as would be readily apparent to one of ordinary skill in the art. For instance, features illustrated or described as part of one implementation can be used with another implementation to yield a still further implementation. Thus, it is intended that the present disclosure cover such alterations, variations, and equivalents.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (a-a, a-a-a, a-a-b, a-a-c, a-b-b, a-c-c, b-b, b-b-b, b-b-c, c-c, and c-c-c or any other ordering of a, b, and c).

While various embodiments of the disclosure are described in the foregoing description and shown in the drawings, it is to be distinctly understood that this disclosure is not limited thereto but may be variously embodied to practice within the scope of the following claims. From the foregoing description, it will be apparent that various changes may be made without departing from the spirit and scope of the disclosure as defined by the following claims.

What is claimed is:

1. An ultrasound system comprising:
an ultrasound scanner configured to transmit ultrasound at a patient anatomy and receive reflections of the ultrasound from the patient anatomy;
a memory configured to store instructions and ultrasound data generated based on the reflections of the ultrasound; and
a processor system coupled to the memory and configured to execute the instructions to:
receive, from the ultrasound scanner, the ultrasound data;
generate quantum-formatted ultrasound data comprising the ultrasound data encoded into a quantum format, the quantum format:
configured to be read by a quantum computing system; and
based on a structure of one or more qubits of the quantum computing system; and
transmit the quantum-formatted ultrasound data to the quantum computing system.

2. The ultrasound system of claim 1, wherein the processor system is further configured to execute the instructions to:
receive, from the quantum computing system, encoded quantum-processed ultrasound data based on the quantum-formatted ultrasound data; and
decode the encoded quantum-processed ultrasound data.

3. The ultrasound system as described in claim 2, wherein the processor system is further configured to execute the instructions to:
embed a training object into the ultrasound data;
generate, based on the decoded quantum-processed ultrasound data and the embedded training object, a first inference for the training object; and
determine, based on the training object, a score for the first inference.

4. The ultrasound system as described in claim 3, wherein the processor system is further configured to execute the instructions to:
generate, based on the decoded quantum-processed ultrasound data, a second inference for the patient anatomy; and
determine, based on the score for the first inference, to accept or reject the second inference for the patient anatomy.

5. The ultrasound system as described in claim 3, wherein the processor system is further configured to execute the instructions to generate, based on at least one random variable, the training object.

6. The ultrasound system of claim 2, wherein the encoded quantum-processed ultrasound data comprises the quantum-formatted ultrasound data processed by one or more quantum operations, wherein:
the one or more quantum operations are configured to transform one or more initial states of the one or more qubits into one or more transformed states of the one or more qubits; and
the decoded quantum-processed ultrasound data is based on the one or more transformed states of the one or more qubits.

7. The ultrasound system of claim 6, wherein:
the structure of the one or more qubits of the quantum computing system comprises an entanglement of at least two of the one or more qubits of the quantum computing system; and
at least one of the one or more quantum operations transforms the two or more entangled qubits of the quantum computing system in a single transformation operation.

8. The ultrasound system of claim 2, wherein the processor system is further configured to execute the instructions to generate, based on the decoded quantum-processed ultrasound data, an image.

9. The ultrasound system of claim 8, wherein the decoded quantum-processed ultrasound data comprises a quantum inference based on the ultrasound data.

10. The ultrasound system as described in claim 9, further comprising a display device, wherein the processor system is further configured to execute the instructions to implement a machine-learned model configured to generate, based on the decoded quantum-processed ultrasound data, at least one of a classical inference and another image, and the display device is implemented to display a comparison of the quantum inference and the classical inference or a comparison of the image and the other image.

11. The ultrasound system as described in claim 9, wherein the quantum computer is configured to implement a quantum machine-learned model to generate the quantum inference.

12. The ultrasound system of claim 1, wherein the ultrasound data based on the reflections of the ultrasound from the patient anatomy is image data, the image data comprising information on one or more of intensity values, location values, or color values configured to construct one or more pixels from the image data.

13. The ultrasound system of claim 12, wherein the encoding of the image data into the quantum format comprises mapping one or more of the intensity values, the location values, or the color values to one or more of an amplitude value or a phase value for the one or more qubits of the quantum computing system.

14. The ultrasound system of claim 1, wherein the one or more qubits of the quantum computing system are virtual qubits instantiated on a classical computing system.

15. An ultrasound system comprising:
an ultrasound scanner configured to transmit ultrasound at a patient anatomy and receive reflections of the ultrasound from the patient anatomy;
a transceiver;
a display device; and
a processor system coupled to the ultrasound scanner, the display device, and the transceiver, and configured to:
generate ultrasound data based on the reflections of the ultrasound;
embed a training object into the ultrasound data;
generate quantum-formatted ultrasound data by encoding the ultrasound data into a quantum format, the quantum format:
configured to be read by a quantum computing system; and
based on a structure of one or more qubits of the quantum computing system;
cause the transceiver to transmit the quantum-formatted ultrasound data to the quantum computing system;
cause the transceiver to receive, from the quantum computing system, encoded quantum-processed ultrasound data based on the quantum-formatted ultrasound data;
decode the encoded quantum-processed ultrasound data;
generate, based on the decoded quantum-processed ultrasound data, a first inference for the training object;
generate, based on one or more of the ultrasound data, the decoded quantum-processed ultrasound data, or the first inference, an ultrasound image; and
cause the display device to display the ultrasound image and a visual representation of the first inference.

16. The ultrasound system as described in claim 15, wherein the processor system is further configured to:
generate, based on the decoded quantum-processed ultrasound data, a second inference for the patient anatomy
determine, based on the training object, a score for the first inference;
determine, based on the score for the first inference, a reliability measure for the second inference for the patient anatomy; and
cause the display device to display a visual representation of the reliability measure.

17. The ultrasound system as described in claim 16, wherein the processor system is further configured to:
compare the reliability measure to a threshold value and determine, based on the comparison, to accept or reject the second inference for the patient anatomy; and
based on the determination being to accept the second inference for the patient anatomy, populate a medical worksheet with an indication of the second inference for the patient anatomy.

18. The ultrasound system as described in claim 17, wherein the processor system is further configured to cause the transceiver to send the medical worksheet to a medical archiver.

19. The ultrasound system as described in claim 17, wherein the processor system is further configured to, based on the determination being to reject the second inference for the patient anatomy:
embed an additional training object into additional ultrasound data;
generate additional quantum-formatted ultrasound data comprising the additional ultrasound data encoded into the quantum format; and
cause the transceiver to transmit the additional quantum-formatted ultrasound data to the quantum computing system.

20. An ultrasound system comprising:
an ultrasound scanner configured to transmit ultrasound at a patient anatomy and receive reflections of the ultrasound from the patient anatomy;
an image generator configured to generate an ultrasound image from the reflections of the ultrasound; and
a data formatter configured to format the ultrasound image such that it is:
able to be read by a quantum computing system; and
based on a structure of one or more qubits of the quantum computing system, the quantum computing system configured to implement a quantum machine-learned model that is configured to generate, based on the formatted ultrasound image, an inference for the patient anatomy.
